(19) United States
(12) Patent Application Publication
(10) Pub. No.: US 2011/0118171 A1
(43) Pub. Date: May 19, 2011

(54) PROCESS FOR PRODUCING FATTY ACID AND ACTIVE INGREDIENT EXTRACTS, AND FATTY ACIDS AND ACTIVE INGREDIENTS THEMSELVES

(75) Inventor: Hermann-Josef Wilhelm, Kalkar (DE)

(73) Assignee: CONPOWER ENERGIEANLAGEN GMBH & CO. KG, GRÄFELFING (DE)

(21) Appl. No.: 12/988,848

(22) PCT Filed: Apr. 9, 2009
(86) PCT No.: PCT/EP2009/002677

§ 371 Date: Dec. 23, 2010

(30) Foreign Application Priority Data
Apr. 21, 2008 (DE) 10 2008 019 954.0
Sep. 1, 2008 (DE) 10 2008 045 281.5

(51) Int. Cl. A61K 38/02; C11B 1/10; A61K 31/201; A61K 31/4172; C09B 61/00; C09K 3/00

(52) U.S. Cl. 514/1.1; 554/8; 514/560; 514/400; 8/646; 252/182.12

(57) ABSTRACT

A method for producing especially oleic acid and fatty acid extracts, and oleic acids and fatty acids themselves. In order to obtain active ingredients, especially oleic acids or fatty acids, from a noninvasive plant in large amounts, without the use of genetic manipulation processes, for reasons relating to agricultural laws, the oleic acid or fatty acid extracts are obtained from the biological material, i.e. the plant material, of the novel plant variety CANDY (CPVO 2007/1958).

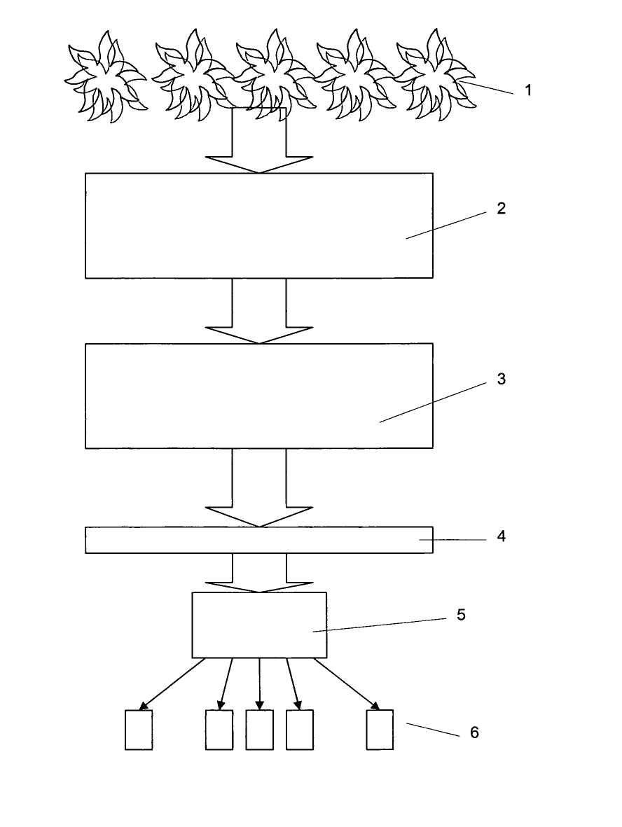

The invention relates to a process in particular for producing oleic acid or fatty acid extracts, and oleic acids or fatty acids themselves, according to the characterizing clause of patent claims 1 and 11.

An example of a plant from the genus Polygonacea is Polygonum sachalinense. Various kinds of ingredients are known from it, for example substances from the anthraquinone group, such as emodin and physcion. Furthermore, its further spectrum of active substances, with regard to the amount of valuable substances, is unspectacular. Yet even for the case of the occurrence of valuable ingredients, the known polygonaceae are extremely invasive plants, which cannot be grown in plantations, because they very quickly spread rampantly with root runners and seeds. For this reason controlled cultivation for obtaining active substances is not possible.

The invention is based on the task of obtaining active substances, in particular oleic acids or fatty acids, in large amounts from a noninvasive plant. For reasons of agricultural law, this must be achieved without using methods of genetic engineering.

This task is solved according to the invention in a method of the generic kind, by the characterizing features of claim 1.

Further advantageous embodiments are presented in the dependent claims 2 to 10.

With respect to an extract of active ingredient or raw material, this task is solved according to the invention by the characterizing features of claims 11, 12 and 13.

Various other raw materials are presented in the other claims.

The new cultivar (Candy) CPVO 2007/1958 is obtainable from 2E Erneuerbare Energie GmbH, Brunhamstrasse 21, 80249 Munich. It is sold there under the brand names CANDY, or Igniscum-Candy. The plant genus from which the new plant originates is the Polygonacea genus. Further information on the plant can be seen under the aforementioned publication number on the website of the CPVO (Community-Plant-Variety-Office) in Angers, France.

Thus, in the prior art essentially two branches of this genus are examined, namely Reynoutria sachalinensis and Reynoutria japonica, also called Polygonum cuspitatum.

Apart from the active substances known from that, high sugar contents and high contents of oleic or fatty acid are not known. Moreover, the species or varieties Sachalinense and Japonica are extremely invasive, via their flowers and fruits and mainly via their very aggressively and wildly growing roots.

The stated new cultivar is based, however, on a new plant that mutated at first spontaneously from these strains in nature, and on observing this mutation was isolated immediately. This mutation at first included all biological structures. However, in order to reduce the invasiveness of the plant, further breeding selection was applied, which led to sterility (empty fructescences) and to greatly reduced root development. These properties were then further developed in the plant, which was then only propagated vegetatively (propagation by root rhizomes) especially by deliberate selection of these properties. That is, these new properties were stabilized by breeding and breeding selection.

A change, which became more and more pronounced, was also observed in the physique of the plant, but in particular a significant reactivity to light, by a significant “reversible” color change, especially in red tones. Moreover, during these systematic breeding steps that were described, a change in the chemistry of the plant was detected. In the subsequent selections during propagation of further generations, certain groups of substances were intentionally expressed, which led to the hitherto unknown amounts of various oleic and/or fatty acids.

Some components of this are completely unknown in such high concentrations in this plant genus. The unusually high sugar contents resulting from breeding are also significant.

What is particularly important is that the new plant contains the aforesaid various oleic acids or fatty acids and moreover in significantly high concentrations in the plant material, without it being altered by genetic engineering. This is of particular technical but also economic and ecological importance.

The new plant properties resulting from the aforesaid selective breeding steps have now become stable and varietally pure. A “reversion” to old genetically original properties such as invasiveness, or a significant reduction in the contents of substances established, has not been observed. That is, without further selection, the plant properties and the spectrum of ingredients are stable, and can be generated by simple vegetative propagation with causal success and repeatably. Said plant is therefore available now.

The core of the process-related invention is that the oleic or fatty acid extracts are obtained from the biological material, i.e. the plant material of the new cultivar CANDY (CPVO 2007/1958). Thus, a source is now available, which was not genetically modified for this, but was obtained by conventional breeding so that growing restrictions are avoided ab initio. There is also an extremely important aspect. The stated new cultivar gives yields of approx. 300 tonnes wet weight or more per hectare. This leads to approx. 40 tonnes dry weight per hectare or more. This example clearly shows what amounts of the fatty acids stated below are made available by this new cultivar. The result is a total fat content of 3 to 6% and even much more in the harvested plant material. It depends on the harvesting time, soil quality and application of fertilizer.

In a further advantageous embodiment it is stated that the oleic or fatty acid extracts are obtained from the dry weight/dry matter of the plant or the dried biomass of the new cultivar CANDY (CPVO 2007/1958). The ingredients/oils/fatty acids specified in more detail below are obtained more easily from the dry mass of the plant (i.e. from the dried plant material).

In a further advantageous embodiment it is shown that the following saturated fatty acids are extracted
palmitic acid (C16:0) and/or
margaric acid (C17:0) and/or
stearic acid (C18:0) and/or
arachidic acid (C20:0) and/or
behenic acid (C22:0) and/or
lignoceric acid (C24:0) and/or
hexanoic acid (C6:0) and/or
octanoic acid (C8:0) and/or
capric acid (C10:0) and/or
lauric acid (C12:0) and/or
myristic acid (C14:0) and/or
pentadecanoic acid (15:0).

The stated fatty acids are given their trivial names rather than their chemical names, because literature searches are better using trivial names. Thus, these are saturated fatty acids. Palmitic acid and arachidic acid are especially significant, each being represented with up to approx. 20% of the total fat content in the dry weight.

Palmitic acid, also called hexadecanoic acid, frequently occurs in plant and animal fats. For example, it is represented at up to 46% in palm oil. However, this relates to palm oil already extracted, and not the proportion in the dry mass from which palm oil is obtained. Therefore the proportion of 20% of the total fat in the present Polygonum plant that was modified by breeding techniques is really substantial, and accordingly not to be expected for a polygonum plant.

Furthermore, the new cultivar stated in claims 1 and 2 can be grown with far fewer problems than the usual plants for palm oil.

Moreover, the new cultivar, as already mentioned, has lost its invasiveness as a result of breeding and can therefore be grown easily.

The stated plant dry matter also has a high content of arachidic acid. The proportion of up to approx. 20% of the total fat content of the dry weight is unusually high for this fatty acid.

These two items of information and the information presented on achievable yields per hectare clearly show in what large amounts these fatty acids can be obtained from this new cultivar.

All the other stated saturated fatty acids are present in lower concentrations between 0.1 and 3.0% of the total fat content of the dry weight.

In a further embodiment, the at least mono-unsaturated fatty acids are stated, namely
myristoleic acid (C14:1) and/or
palmitoleic acid (C16:1) and/or
oleic acid (C18:1) and/or
linoleic acid (C18:2) and/or
linolenic acid (C18:3) and/or
eicosadienoic acid (C20:2) and/or
erucic acid (C22:1) and/or
eicosatrienoic acid (20:3) and/or
arachidonic acid (C20:4) and/or
nervonic acid (C24:1).

Of these, eicosatrienoic acid C20:3 is the most represented, namely with up to approx. 17% of the total fat content of the dry weight of the stated plant. This is unusually high. In the case of eicosatrienoic acid, this is already the chemically common designation, or also dihomogammalinolenic acid. This is triply unsaturated and is one of the omega-6 fatty acids.

The mono-unsaturated oleic acid C18:1 delta-9-octadecenoic acid, which is one of the omega-9 fatty acids, is present at up to approx. 10%. It is also present in palm oil and groundnut oil. But this too should be assessed with the aforementioned yields. It can be seen that this fatty acid is also made available in considerable amounts by the stated new cultivar.

At approx. 8.5%, the doubly unsaturated linoleic acid C18:2 occurs in the plant as 9Z, 12Z- or delta-9-cis-, delta-12-cis-octadecadienoic acid. It is one of the omega-6 fatty acids.

Alpha- or gamma-linolenic acid is represented as C18:3; alpha-linolenic acid is one of the omega-3 fatty acids and gamma-linolenic acid is one of the omega-6 fatty acids.

Quite especially, reference to the achievable yields of the stated plant has an essential role for the high availability of this substance as raw material.

All the other stated at least mono-unsaturated fatty acids are represented at 0.1 to 1.5% of the total fat content in the dry weight of said plant. Once again, however, even in single-digit percentages the substances are ultimately available in a notable amount because of the high yields that are achievable.

In a further advantageous embodiment it is envisaged that all of the fatty acids are obtained in a first step as mixed fatty acid extract from said new plant, and then only from the oleic or fatty acid mixture, the various stated fatty acids being separated from one another in a chemical separation stage. With this step it is possible to avoid, in direct extraction of a single fatty acid from the plant, the other fatty acids only being extractable expensively, or no longer at all, or only in an unwanted form.

The production first of a total oil or fat extract as a mixture also has appreciable production engineering advantages. Thus, the actual extraction can be performed in an essentially continuous process—customary oleic or fatty acid extraction processes can be applied, as presented below as an example—and mass splitting to the individual oleic and/or fatty acids only takes place afterwards.

In a further advantageous embodiment it is envisaged that immediately after harvesting the plant material or biomass, the harvested material is separated and dried to prevent unintentional initiation of fermentation or is prepared for the extraction. Owing to the high proportions of raw protein and fatty acids, the harvested plant material of said plant starts to ferment immediately, and heat is generated. When the material is in a stack, this heat gives rise to a quasi-feedback acceleration of fermentation. This must be avoided, in favor of the valuable ingredients stated hereunder. This can be achieved by separating the material immediately after harvesting and processing it further, or separating and drying it.

For preparation of the oleic or fatty acid extract, it is naturally also possible to proceed selectively immediately, since the fatty acids differ from one another in their polarity and in their hydrophobic or possibly also hydrophilic property.

Basically, organic solvents are useful for this. These can be alcohols such as methanol or ethanol, or also aromatic solvents; acetone is also suitable for certain fatty acids. Hexane C₆H₁₄ is also suitable as a solvent. The plant material is reduced to a specified particle size in usual mechanical comminution and then mixed with the solvents. The processes used are known from the prior art.

The present special feature, however, is that a considerable amount of plant material, which results from the enormous yields of said plant, is treated in a large-scale process, so that first a mixture of the stated fatty acids is obtained. Subsequently, the particular fatty acids required can then be separated from the overall fatty acid extract obtained. This therefore also has logistic advantages within the production process.

In a further advantageous embodiment, the following ingredients can then be obtained from the rest of the substrate, i.e. the rest of the plant material:

Process according to one of the preceding claims,
raw protein
sugar
sucrose
fructose
glucose.

Raw protein is also present at up to approx. 13% in the dry weight of the stated new plant. Relative to the possible yields stated at the beginning, of a good 30 tonnes dry weight per hectare per year, almost 4 tonnes per hectare per year of raw protein alone can be obtained.

Sugar is present in the dry weight at up to 10.5%, so that a value is reached going at least in the direction of sugar cane. This is really unusual for a polygonum plant and was not to be expected. The sugar content also has an important role, owing to the enormous yields. Along with the simple sugar analysis, however, fructose, glucose and sucrose also occur. Overall, a total sugar content of up to approx. 15% is obtained. The following miscellaneous substances, in particular amino acids, are present in proportions between 0.2% and 2.0%. Both essential and nonessential amino acids are present.

The essential amino acids from said plant include:
isoleucine
leucine
lysine
methionine
phenylalanine
threonine
valine

Therefore, surprisingly, it contains 7 out of the total of 8 essential amino acids from the series of the canonical amino acids. Leucine is represented at 1.11%. All the others are in the range from 0.3 to 1%.

Once again, the established chemical plant property plays a significant role, in view of the yields that can be achieved.

The nonessential amino acids from said plant include:
cystine
arginine
aspartic acid
glutamic acid
glycine
histidine
serine
tyrosine

These are present in the plant with values between 0.2 and 1.5%.

Another ingredient is choline, which is present at 288 milligrams per kg dry weight.

Once again, as with all ingredients of said new plant CANDY (CPVO 2007/1958), what is remarkable is the range of ingredients, which is also quantitatively important in view of the yields achieved with the plant. This also applies to ingredients that at first are only present at low concentrations, for example choline.

It should be noted in particular that said plant does not display any antibiotic activity in the analyses. This offers the important advantage that little if any pharmacological impurities are to be expected in the extraction processes, if the plant is treated according to the supplier's guarantees and instructions regarding requirements on planting and choice of soil and fertilizers.

Nevertheless, during subsequent utilization of the plant material at the respective manufacturers it is always necessary in each case to clarify by analysis whether ingredients of pharmacological importance are contained, which might possibly also occur because of certain trace substances in the soil of the growing area.

In another advantageous embodiment it is envisaged that active ingredient extracts are obtained from the plant and they are separated according to the different groups of substances, such as fats, amino acids etc. These then form the raw materials for various applications.

For instance as food supplement raw material, as cosmetic raw material, as pharmaceutical raw material, as raw material for plant protection products, as raw material for plant auxiliary products, as raw material for biogas additive and as raw material for dyes.

Owing to the extremely wide spectrum of ingredients of said plant, there is also this wide spectrum of resultant raw materials.

Because of the aforementioned high yields, fatty acids in particular are available in large amounts. This also applies to raw protein and other substances.

One aspect of the raw material basis relates to biogas raw material. An additive from this plant promotes the gas production rate of biomass, sometimes very significantly.

Moreover, preliminary tests have shown that the new plant, as principal substrate in biogas installations, produces remarkable gas production rates. The plant or its active ingredient extracts are therefore good additives for the generation of higher gas production rates in biogas installations.

The table shows an example of quantitative data for the range of ingredients of said plant.

The values are selected in the data in such a way that the figure assigned in each case to a substance represents a possible “maximum value”. The individual values are basically not always attained, but vary mutually so that the total is not above 100% of the dry weight or, in the case of fats, the total fat content.

The tables contain the corresponding values. To differentiate them, the fatty acids are given along with their respective structural formula. For example C20:0 denotes fatty acid with 20 carbon atoms and the zero stands for saturated. In the case of for example C18:3, the 3 stands for triply unsaturated.

All the information is stated as percentage (%) of dry matter. The abbreviation DM is used for this. Formally, the notation used is

Mₜₒₜ=M_{w}+M_{D}

However, Mₜₒₜ describes just the moist harvested total biomass, which contains the mass of water M_{W} and the mass of dry matter M_{D}.

For the values used as a basis here, this is already the dry matter or dry weight M_{D} (denoted DM in analyses) of the harvested and then dried biomass, in this case of the new cultivar CANDY (CPVO 2007/1958).

The total spectrum of ingredients of said plant is striking. Owing to the enormous yields, namely 300 tonnes of fresh mass per hectare, which after drying still gives a dry weight of about 30 to 50 tonnes/hectare, each of the stated substances is of interest because of the biomass that is available.

The range of fatty acids, as well as the range of amino acids, in these percentages of the dry matter, is really unusual for polygonum plants and is exclusively a result of the use according to the invention of this new plant. Such high fatty acid contents in a polygonum plant have not been reported in the literature before.

| | |
|---|---|
| Raw protein | 13.6 |
| Sugar | 10.5 |
| Carbon | 44 |
| Hexanoic acid (C8:0) | <0.1 |
| Octanoic acid (C8:0) | 0.2 |
| Capric acid (C10:0) | 0.1 |
| Lauric acid (C12:0) | 2.0 |
| Myristic acid (C14:0) | 2.4 |
| Myristoleic acid (C14:1) | <0.1 |
| Pentadecanoic acid (C15:0) | <0.1 |
| Palmitic acid (C16:0) | 19.1 |
| Palmitoleic acid (C16:1) | 1.4 |
| Margaric acid (C17:0) | <0.1 |
| Stearic acid (C18:0) | 3.0 |
| Oleic acid (C18:1) | 10.2 |
| Linoleic acid (C18:2) | 8.8 |
| Arachidic acid (C20:0) | 20.1 |
| Eicosenoic acid (C20:0) | <0.1 |
| Linolenic acid (C18:2) | 13.2 |
| Eicosadienoic acid (C20:2) | <0.1 |
| Behenic acid (C22:0) | 1.6 |
| Erucic acid (C22:1) | <0.1 |
| Eicosatrienic acid (C20:3) | 16.9 |
| Arachidonic acid (C20:4) | <0.1 |
| Lignoceric acid (C24:0) | 1.1 |
| Nervonic acid (C24:1) | <0.1 |
| Lysine | 0.78 |
| Cystine | 0.17 |
| Methionine | 0.28 |
| Threonine | 0.58 |
| Arginine | 0.71 |
| Aspartic acid | 1.19 |
| Choline | 288 mg/kg |
| Fructose | 1.88 |
| Glucose | 1.93 |
| Glutamic acid | 1.48 |
| Glycine | 0.77 |
| Histidine | 0.35 |
| Isoleucine | 0.59 |
| Leucine | 1.11 |
| Phenylalanine | 0.72 |
| Sucrose | 0.35 |
| Serine | 0.62 |
| Tyrosine | 0.46 |
| Valine | 0.7 |

The drawing shows the stepwise processing of the new plant varieties for the example of a total mixed fatty acid extract or a mixed oil extract. The fresh mass 1, in this case said new cultivar Candy, is harvested as the whole plant above the root ball and then in the next stage 2 is separated and dried to prevent uncontrolled fermentation. Then the dry matter is submitted to a processing stage 3 for production of the extracts. This can be a chemical processing stage, or if possible a combination of oil mill of the conventional type together with feed of chemical extractants/solvents.

The extract obtained 4 can then already be the total oil, or the mixed oil or mixed fat with all fatty acid constituents as a mixture.

The chemical treatment of the fatty acid to fat or of the oleic acid to oil can take place separately or even simultaneously in the extraction step 3.

This is followed by mass-related separation 5 of the individual fatty acid or oleic acid constituents, or fats or oils.

The individual separated components 6 are then sent to the individual specific uses.

1-20. (canceled)

21. A method for producing oleic acid extracts, the method comprising the following step:
obtaining oleic or fatty acid extracts from biological plant material of a cultivar CANDY.

22. The method according to claim 21, which further comprises obtaining the oleic or fatty acid extracts from a dry mass of a plant or a dried biomass of the cultivar CANDY.

23. The method according to claim 22, which further comprises immediately after harvesting the plant material or the biomass, separating and drying or preparing the harvested material for extracts, to avoid unintentional initiation of fermentation.

24. The method according to claim 21, which further comprises extracting the following saturated fatty acids:
palmitic acid (C16:0) and/or
margaric acid (C17:0) and/or
stearic acid (C18:0) and/or
arachidic acid (C20:0) and/or
behenic acid (C22:0) and/or
lignoceric acid (C24:0) and/or
hexanoic acid (C6:0) and/or
octanoic acid (C8:0) and/or
capric acid (C10:0) and/or
lauric acid (C12:0) and/or
myristic acid (C14:0) and/or
pentadecanoic acid (15:0).

25. The method according to claim 21, which further comprises extracting the following, at least mono-unsaturated fatty acids:
myristoleic acid (C14:1) and/or
palmitoleic acid (C16:1) and/or
oleic acid (C18:1) and/or
linoleic acid (C18:2) and/or
linolenic acid (C18:3) and/or
eicosadienoic acid (C20:2) and/or
erucic acid (C22:1) and/or
eicosatrienoic acid (20:3) and/or
arachidonic acid (C20:4) and/or
nervonic acid (C24:1).

26. The method according to claim 24, which further comprises obtaining total fatty acids in a first step as mixed oil extract from the plant, and then separating the various fatty acids from one another in a chemical separation stage.

27. The method according to claim 25, which further comprises obtaining total fatty acids in a first step as mixed oil extract from the plant, and then separating the various fatty acids from one another in a chemical separation stage.

28. The method according to claim 21, which further comprises obtaining the following substances from the plant material:
raw protein (XP)
sugar (XZ)
sucrose
fructose
glucose.

29. The method according to claim 21, which further comprises obtaining the following amino acids from the plant material:
arginine and/or
aspartic acid and/or
cysteine and/or
glutamic acid and/or
glycine and/or
histidine and/or
isoleucine and/or
leucine and/or
lysine and/or
methionine and/or
phenylalanine and/or
serine and/or
tyrosine and/or
valine.

30. The method according to claim 21, which further comprises obtaining choline from the new cultivar.

31. The method according to claim 21, which further comprises obtaining active ingredient extracts or fatty acid extracts or mixed extracts from a mixture of various fatty acids with an organic solvent, and then obtaining the extract after evaporation of the solvents.

32. The method according to claim 31, which further comprises selecting the organic solvent as at least one solvent from the group consisting of methanol, ethanol, aromatic solvents, acetone and a mixture of the solvents.

33. An active ingredient or raw material extract from a cultivar CANDY, the extract comprising:
a mixture of at least two components of the following saturated and/or unsaturated fatty acids:
palmitic acid (C16:0) and/or
margaric acid (C17:0) and/or
stearic acid (C18:0) and/or
arachidic acid (C20:0) and/or
behenic acid (C22:0) and/or
lignoceric acid (C24:0) and/or
hexanoic acid (C6:0) and/or
octanoic acid (C8:0) and/or
capric acid (C10:0) and/or
lauric acid (C12:0) and/or
myristic acid (C14:0) and/or
pentadecanoic acid (15:0), and/or
myristoleic acid (C14:1) and/or
palmitoleic acid (C16:1) and/or
oleic acid (C18:1) and/or
linoleic acid (C18:2) and/or
linolenic acid (C18:3) and/or
eicosadienoic acid (C20:2) and/or
erucic acid (C22:1) and/or
eicosatrienoic acid (20:3) and/or
arachidonic acid (C20:4) and/or
nervonic acid (C24:1).

34. The active ingredient or raw material extract according to claim 33, which further comprises the following further components obtained from the cultivar CANDY:
raw protein (XP)
sugar (XZ)
sucrose
fructose
glucose.

35. The active ingredient or raw material extract according to claim 33, which further comprises the following further components obtained from the cultivar CANDY:
arginine and/or
aspartic acid and/or
cysteine and/or
glutamic acid and/or
glycine and/or
histidine and/or
isoleucine and/or
leucine and/or
lysine and/or
methionine and/or
phenylalanine and/or
serine and/or
tyrosine and/or
valine
choline.

36. A food supplement raw material, comprising the active ingredient extract according to claim 33.

37. A cosmetic raw material, comprising the active ingredient extract according to claim 33.

38. A pharmaceutical raw material, comprising the active ingredient extract according to claim 33.

39. A raw material for plant protection products, comprising the active ingredient extract according to claim 33.

40. A raw material for plant auxiliary products, comprising the active ingredient extract according to claim 33.

41. A raw material for biogas additive, comprising the active ingredient extract according to claim 33.

42. A raw material for dyes, comprising the active ingredient extract according to claim 33.
